(19) United States
(12) Patent Application Publication
(10) Pub. No.: US 2012/0277612 A1
(43) Pub. Date: Nov. 1, 2012

(54) SYSTEMS FOR INTRAVENOUS DRUG MONITORING

(75) Inventor: Bo Li, Rexford, NY (US)

(73) Assignee: GENERAL ELECTRIC COMPANY, Schenectady, NY (US)

(21) Appl. No.: 13/173,734

(22) Filed: Jun. 30, 2011

Related U.S. Application Data
(60) Provisional application No. 61/479,428, filed on Apr. 27, 2011.

(51) Int. Cl. A61B 5/097; A61M 5/168

(52) U.S. Cl. 600/532; 604/66

(57) ABSTRACT

A system for monitoring a concentration of an anesthetic drug using a patient's breath is provided. The system comprises a sampling subsystem for processing the patient's breath to form a breath sample, one or more sensors to measure drug concentration in the breath sample, one or more sensors to measure a concentration of gases in the breath sample; and one or more microprocessors for determining a concentration of the drug in a plasma of the patient using a transfer function and the concentration of the drug in the breath sample. A system for monitoring propofol concentration in patient's breath sample is also provided.

This non-provisional application claims the benefit of priority under 35 U.S.C. §119(e) to U.S. Provisional Patent Application Ser. No. 61/479428, filed Apr. 27, 2011, which is herein incorporated in its entirety by reference.

### TECHNICAL FIELD

The invention relates generally to a system for intravenous drug monitoring, and more specifically to a system for intravenous anesthesia drug monitoring.

### BACKGROUND

Intravenous anesthetic agents are typically short acting agents. The intravenous anesthetic agents are generally used in induction and maintenance phase of anesthesia. Based on the rapid distribution and metabolism of the anesthetic agents in patients' bodies, the anesthetic must be re-dosed frequently to ensure the anesthesia depth and the success of surgery. The control of the anesthesia amount is mainly based on the prediction of pharmacokinetic models. However, the pharmacokinetic models are not able to compensate the individual difference of each patient's physical characteristics, and may lead to determine a dose which may be an under-dose or overdose for the patient, either resulting in early wakeup during procedure or causing side effects. Therefore, precise and real-time detection of anesthetic concentration in plasma is greatly needed to improve the quality of anesthesia monitoring.

Different approaches are available to monitor patients under anesthesia procedures. These methods can be categorized into direct measurement of anesthetic drug concentration in blood and indirect measurement by monitoring a patient's conscious level, in addition to normal physiological parameters such as oxygen saturation, blood pressure, or heart rate. The anesthetic drugs may be detected in plasma or breath samples. Monitoring of anesthetic drug concentration in plasma or breath may provide a better protection to patients than other conventional methods. The depth of anesthesia for a known concentration of drug in plasma is less variable; however, there is a significant interpatient variability in the drug concentration in plasma achieved with a known dose of anesthetic drug. The direct measurement of drug in plasma is invasive, time consuming and expensive. In contrast to direct method, an indirect breath based approach would be non-invasive, and provide continuous monitoring, faster response times and lower costs.

Therefore, a device for monitoring a plasma concentration of intravenously delivered anesthetic drug by measuring the drug vapour concentration from exhaled breath is highly desirable.

### BRIEF DESCRIPTION OF THE INVENTION

In one embodiment, a system for monitoring a concentration of an anesthetic drugs using a patient's breath comprises a sampling subsystem for processing the patient's breath to form a breath sample, one or more sensors to measure drug concentration in the breath sample, one or more sensors to measure a concentration of gases in the breath sample; and one or more microprocessors for determining a concentration of the drug in a plasma of the patient using a transfer function and the concentration of the drug in the breath sample.

In another embodiment of the system for monitoring a concentration of propofol using a patient's breath comprises a sampling subsystem for processing the patient's breath to form a breath sample, one or more sensors to measure propofol concentration in the breath sample, one or more sensors to measure a concentration of gases in the breath sample; and one or more microprocessors for determining a concentration of the propofol in a plasma of the patient using a transfer function and the concentration of the propofol in the breath sample.

### BRIEF DESCRIPTION OF THE DRAWINGS

These and other features of embodiments of the invention will be more readily understood from the following detailed description of the various aspects of the invention taken in conjunction with the accompanying drawings that depict various embodiments of the invention, in which:

FIG. 1 is a schematic diagram of an embodiment of a device for intravenous anesthetic drug monitoring according to one aspect of the invention.

### DETAILED DESCRIPTION OF THE INVENTION

One or more examples of a system are adapted for detecting a concentration of an anesthetic drug in plasma during general or total anesthesia operation. Anesthetic drugs may be administered parenterally, sublingually, transdermally, by intravenous bolus, and by continuous infusion. Anesthetic agents may be administered in an amount for analgesia, conscious sedation, or unconsciousness as per its known dose. The concentration of the anesthetic agent in exhaled breath reflects the condition of a patient under the anesthetic drug treatment. For example, in case of higher concentration of drug in blood stream provides information on accumulation of drugs in the blood stream, which may cause a deep level of anesthesia. In another example, if the concentration of anesthetic drug in the blood stream decreases with time, this may possibly lead to inadequate anesthesia and premature emergence.

To more clearly and concisely describe and point out the subject matter of the claimed invention, the following definitions are provided for specific terms, which are used in the following description and the appended claims. Throughout the specification, use of specific terms should be considered as non-limiting examples.

As used herein, the term “module” refers to software, hardware, or firmware, or any combination of these, or any system, process, or functionality that performs or facilitates the processes described herein.

One embodiment of the system for monitoring a concentration of an anesthetic drug using a patient's breath comprises a sampling subsystem for processing the patient's breath to form a breath sample, one or more sensors to measure drug concentration in the breath sample, one or more sensors to measure a concentration of gases in the breath sample; and one or more microprocessors for determining a concentration of the drug in a plasma of the patient using a transfer function and the concentration of the drug in the breath sample.

In another embodiment, the system for monitoring the concentration of anesthetic drug in plasma is adapted for intravenous drug administration. The intravenously delivered anesthetic drug concentration in plasma is monitored using the system by measuring the drug vapor concentration in a patient's breath. For the intravenous anesthetics application, the quantity of drug required should induce a sufficient depth of anesthesia without accumulating an excessive amount of anesthetic drug.

The system may comprise a breathing circuit, a flow channel, a flow tubing, or an adapter for collecting patient's breath for analysis using the system. The breathing circuit is used to take a breath sample from the patient who is administered one or more drugs intravenously. In one or more embodiments, the breathing circuit may directly be attached to the system for collecting breath followed by processing through the system. In some other embodiments, the breathing circuit may attach to the system indirectly, for example through an adapter.

The configuration of breathing circuit may be different. In some embodiments, the circuit is called a mainstream breathing circuit. In this embodiment, the breathing circuit may be directly connected to the patient's mouth or nose. In a different embodiment, the breathing circuit may be connected to a separate tube, which is directly connected to the patient's mouth or nose, and otherwise referred to as a side stream configuration. In some embodiments, a flow channel or tubing may be attached to, for example, a mouthpiece or nosepiece. The mouthpiece or nosepiece may be used to readily transmit the exhaled breath to the sensor. In another example, the exhaled breath is collected through an adapter at the proximal end of the respiratory track and drawn or pushed through a tubing to the sensor.

In one embodiment, the material for making a breathing circuit, flow channel, tubing or adapter may be selected depending on the surface property of the material. As many of the components of anesthetic drug may be sticky in nature, the material of the breathing circuit, tubing, flow channel or adapter is desirable to have non-sticky in nature. For example, one of the intravenous anesthetic drugs is Propofol, which is a sticky molecule and tends to stick to the surface of the breathing circuit, flow channel, tubing or adapter. The materials of breathing circuit, flow channel, tubing or adapter may include, but are not limited to Teflon, stainless steel, or glass. In some examples, the breathing circuit, flow channel, tubing or adapter may be coated with non-sticking material. In some examples, heated breathing circuit, flow channel, tubing or adapter may also be used to reduce the surface sticking of various components of anesthetic drugs, such as propofol.

The system comprises sampling subsystem for processing the patient's breath to form a “breath sample”. The sampling subsystem comprises a breath sample conduit and a heating element that heats the conduit. In one or more non-limiting examples, the conduit may be a tube, a flow channel, a cylinder, or a pipe. One or more heating elements are attached to the conduit to heat the conduit depending on the operational requirement. The heating element increases the temperature of the conduit to prevent condensation of the anesthetic drug vapor present in the breath flow. Moreover, in some embodiments, the anesthetic drug such as propofol sticks to the conduit at normal temperature. In these embodiments, the heated conduit develops a surface property, so that the anesthetic drug vapor present in the breath sample does not stick to the inner-surface of the conduit. The heating element may be a thin film heater, a heating pad, a solid-state heater, a filament heater, a heating tape, or any heater with a heating element. Generally, heating element maintains a nearly constant temperature of the conduit and prevents water condensation from entering gas, or sticking of the drug vapors to the conduit. In a normal operation, heating element heats the conduit up to about 100° C., but any temperature (e.g., 40 to 50° C.) that is above the temperature of the gas entering the subsystem is sufficient to prevent condensation or surface sticking. The sampling subsystem processes the collected breath from the patient to improve the measurement accuracy of the drug vapor concentration and the processed breath further introduced to the sensors for measuring concentration of anesthetic drug in the breath sample. The sampling subsystem may process a patient's inhaled breath, exhaled breath; or combinations thereof.

In some embodiments of the sampling subsystem comprise two or more devices for filtration, concentration, dilution, desiccation, breath humidity control, normalizing vapor density, breath pressure control, breath temperature control, or breath flow rate control. In one embodiment, the sampling subsystem comprises one or more filters to remove or reduce unwanted substances in the breath sample, such as water vapor, sputum, food particles, or other interfering compounds that may lower the sensitivity and selectivity of the sensors used to detect target drug compounds. In one embodiment, the sampling subsystem may comprise more than one filter depending on the requirement of purification extent of the breath sample. The breath sample may also be mixed or diluted with a known carrier gases to achieve desired pressure or flow rate.

In some embodiments, the system may comprise one or more concentrators those concentrate breath samples. In some embodiments, the breath sample is routed through the pre-concentrator before being passed over the sensor array. By heating and volatilizing the breath (or gases), humidity may be removed. In one embodiment, the exhaled breath is allowed to dry before being exposed to a sensor and the vapor density of each sample of exhaled breath may be normalized before the sensing procedure. One or more dehumidifier may be used to control the vapor density. The humidity in the exhaled breath causes inaccurate detection of various components of the breath sample. When using humidity sensitive devices, the system may employ an electronic nose technology so that a patient may exhale directly into the device with a mean to dehumidify the sample. This is accomplished by using a commercial dehumidifier or a heat moisture exchanger to prevent desiccation of the airway during ventilation with dry gases. One or more water traps may be present in the system to store water condensates from the breath sample. In some embodiments, the patients may exhale breath through their nose which is an anatomical, physiological dehumidifier for normal respiration. In operation, the sensor may be used to identify a baseline spectrum for the patient prior to administration of the drugs. This proves beneficial for the detection of more than one drug if the patient receives more than one drug at a time and possible interference from different foods and odors in the stomach, mouth, esophagus and lungs.

The system comprises one or more of the sensors for detecting anesthetic drugs in the breath sample. The sensors are typically exposed to the breath sample for detecting presence of one or more of the anesthetic drugs. With in-line sampling, the sensor may be placed proximal to the respiratory track directly in the breath stream. One or more of the non-limiting examples of the sensors exposed to the breath sample are flow rate sensors, humidity sensor, pressure sensors, temperature sensors, gas sensors, or drug vapor sensors. In a specific embodiment, the drug vapor sensor may be an intravenous drug vapor sensor.

Some embodiments of the sampling subsystem comprise one or more pressure sensors to monitor the breathing pressure of the breath flow. The sampling subsystem further comprises one or more pressure controllers, wherein the controllers may control the pressure of the breath flow to adjust required pressure while exposing to the system electronics to detect breathing patterns of the patient or provide calibration data. The pressure sensors and pressure controllers may function synergistically for sensing and then controlling pressure depending on its requirement.

In some embodiments, the sampling subsystem comprises one or more temperature sensors to monitor the temperature of the breath sample. The sensing subsystem further comprises one or more temperature controllers to control the temperature of the breath sample and expose to the system electronics for detecting breathing patterns of the patient or provide data calibration or correction. The temperature sensors and temperature controllers may function synergistically for sensing and then controlling temperature depending on the system's requirement. In one embodiment, the temperature controller may be a heating element. Heating element heats the breath flow, if the temperature of the breath is lower than it is required. In addition, heating element and temperature sensor can maintain breath flow at an optimal or constant operating temperature through a temperature feedback control loop to eliminate fluctuation of the baseline of the data calibration due to temperature variation.

In some embodiments, the sampling subsystem further comprises a temperature feedback control circuit. The temperature sensor, temperature feedback control circuit and heating element may be present in an operative association, so that when the temperature of breath sample is different from the desired operational temperature, an error signal is generated based on the temperature sensor's output and a temperature set point. The temperature feedback control circuit activates or turn off the heating element based on the error signal to maintain the temperature of the breath sample to a preset temperature point.

One or more flow sensors may detect the breathing flow rate of the patient. For example, the flow sensor may be used to detect flow rate of the sample at the starting and completion of exhalation process. The sampling subsystem may further comprise a diffuser that regulates a gas flow into the sensor system. Extra sensors may be included in the system, for example, sensors to measure an exhaled carbon dioxide (CO₂), or to measure inhaled and exhaled oxygen (O₂).

In one or more embodiments of the system, the intravenous drug sensor used for measuring concentration of the drug in the breath sample may be a gas sensor or a vapor sensor depending on the drug being monitored. In accordance with one embodiment of the system, the gas sensor is used to detect the concentration of anesthetic drug from exhaled breath of patients during general and total intravenous anesthesia procedure. Measuring concentration of the anesthetic drug in the breath sample is performed using single breath sample or an average of several breath samples. The sensor reading is proportional to the concentration of the anesthetic drug in the breath sample. In one embodiment, the gas sensor measures the vapor concentration of intravenously delivered drug in the patient's exhaled breath. The gas sensor measurement is performed continuously or every few minutes.

In some embodiments, the system may employ more than one drug vapor sensors. One is to measure the inhaled drug concentration, and the other is used to measure the exhaled drug concentration. The difference of the two sensors is used to calculate plasma concentration. The intravenous drug sensors are capable of measuring anesthetic drugs, muscle relaxation drugs, therapeutic drugs, or chemotherapeutic drugs. The intravenous drug sensor may specifically measure the anesthetic drug concentration, such as propofol concentration. In some embodiments, the sensors may also detect metabolic product of the drugs. The possible drug vapor sensors may include, but are not limited to, ion mobility spectrometer, differential mobility spectrometer, polymer based sensor, infrared absorption spectrometer, photoacoustic spectrometer, electrochemical sensors, gravimetric sensors, thermal conductivity sensors, mass spectrometer, or gas chromatography system. For example, electrochemical sensors are employed for the quantification of propofol after chromatographic separations. Propofol is detectable for its oxidation of phenol structure. Furthermore, increasing pH may significantly lower the oxidation potential of propofol. The lower working potential may decrease background signal significantly, since interferences in breath have higher oxidation potentials which may not go down with pH as propofol does, therefore they are not detectable at the low working potential. The sensor may be a single use sensor, wherein the calibration may not be required. In some examples, the sensor may be a re-usable sensor which can be used various times in different operational conditions, where calibration is required for individual operation.

In one or more examples, the drug vapor sensor detects anesthetic drug, such as propofol in patient's breath sample. The calculated anesthetic drug concentration in plasma may trigger an alarm if the value is higher than a preset threshold value. A typical concentration of propofol in the breath of a patient undergoing intravenous anesthesia using propofol is, for example, from 0 ppb to 20 ppb. To measure an accurate amount of drug in the breath sample, the sensors are required to be highly sensitive and selective. The detection limit of the sensor may be in the range of 0.1 ppb to 100 ppb, and the sensor needs to detect the concentration of drug without response to all other potential gas compounds in the breath, for example, acetone, ethanol, isoprene, ammonia, methanol, pentane, or ethane.

In some embodiments, the intravenous drug sensor measures the concentration of one or more drugs in the breath sample. In one or more embodiments, the gas sensors are selected from carbon dioxide sensors (CO₂ sensors), oxygen sensors (O₂ sensors), or drug vapor sensors, or combinations thereof. In some embodiments, the gas sensors detect CO₂ and O₂ concentration from the breath sample. CO₂ concentration is an important parameter for breath measurement. It may be used to detect the end tidal volume of the breath. The end tidal breath is often the most significant part of the entire exhaled breath for analysis. As the end tidal breath typically passes through the gas exchange process in lung and comprises highest CO₂ concentration, a detection of the end tidal breath using a CO₂ sensor is easier. In a normal human subject, this concentration is in a range from about 4% to 5%. Early portions of the breath may contain gas in the dead volume of the air way, which does not participate in the gas exchange in lung. This part of the breath typically is not used to measure drug concentration. In one example, the system electronics for controlling breath sample may use this information and expose the sensors to the end tidal breath for measuring concentration of various components of breath sample. In another example, the sensor electronics comprises the modified drug sensor, which is constantly monitoring the drug concentration in breath. The system electronics may extract the right concentration measurement at the same time when the CO₂ sensor detects the end tidal breath. Similarly, an O₂ sensor may be used for the same purpose as of CO₂ sensor. The CO₂ sensor may also be used to provide real time monitoring of respiration condition of the patient undergoing anesthesia or other procedures. In cases of abnormal CO₂ concentration, typically an alarm is triggered to alert the doctor or other individuals associated with the anesthesia procedure.

In one embodiment, the system is adopted for sampling an end-tidal gas, wherein the samples may be collected throughout the exhalation phase of respiration. In another embodiment, the breath samples are collected at the distal end of the endotracheal tube through a tube with a separate sampling port. The sampling may be improved by allowing a larger sample during each respiratory cycle. Depending on the sample size and detector response time, the breath sample may be collected on successive cycles. The collection of breath from the patient may be a continuous process or an intermittent process. The processing of the patient's breath is performed periodically or continuously. Typically, the drug concentration in plasma during anesthesia procedure may be monitored in real time.

The system further comprises an electronics set up, otherwise referred to as “system electronics”. The system electronics comprises interface circuit to different sensors and actuators, pumps to either receive sensor measurement data or submit signal for actuator, or pump operation. The system electronics further comprises a power supply module. The power supply module is used to supply power to different parts of the whole system. The system electronics may comprise a memory device to store measurement data and calibration data, and may further comprise communication module to transmit and receive data with wired network or wireless network.

The system electronics comprises a microprocessor or a microcontroller to receive, analyze, submit and store measurement and calibration data. The microprocessor determines a concentration of the drug in plasma of the patient using a transfer function and the concentration of the drug in the breath sample. By using the drug concentration in breath, the drug concentration in plasma may be determined accurately using a transfer function. The concentration of drug in plasma may be determined by calculating, computing or correlating the value of drug concentration in plasma using the value of drug concentration in a breath sample and a transfer function. Then the drug concentration in plasma is derived from the anesthetic drug concentration in the breath sample with the use of an appropriate transfer function, which may vary among different situations and for different patients. For example, in one embodiment, the value of transfer function may be dependent on the temperature of patient's body, breathing flow rate, exhaled CO₂ concentration, inhaled and exhaled oxygen concentration, age, gender, weight, height, BMI, or lung function parameters of a patient. The transfer function has an input and an output value. For example, the input of the transfer function may depend on the anesthetic drug concentration in breath and the value of transfer function. The calculated concentration of drug in plasma may be used in several ways. In one embodiment, the input value of the transfer function depends on at least a measured anesthetic drug concentration in the exhaled end tidal breath of a patient. The output value of the transfer function generates the concentration of the delivered drug in plasma. In some examples, the transfer function follows a linear equation or a non-linear equation. In some other examples, the transfer function follows the non-linear equation with a second order or higher order.

The system further comprises a user interface and a display device. The user interface and the display device are operatively coupled to the microprocessors. The user interface is used for user to input data and to collect output data, and also to operate the system. The display device is used to display calibration curves, data generated curves or real time scans. The display device is needed to display required information to the user. The user may change setting of the device depending on display results. Any error shown on the screen may be minimized by changing various parameters.

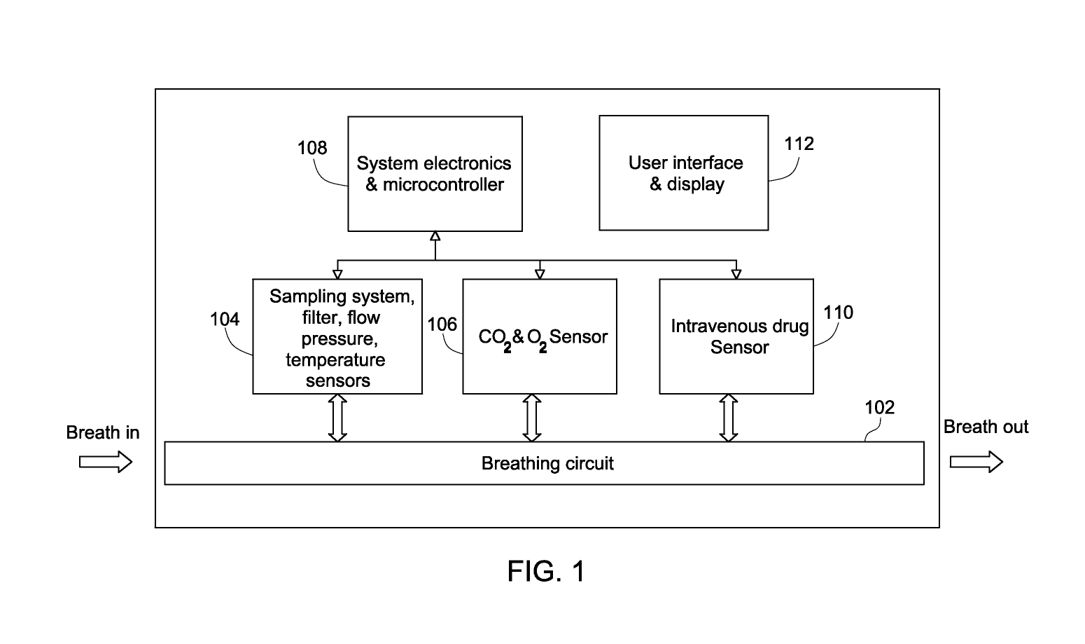

As illustrated in FIG. 1, an exemplary system comprises a breathing circuit 102, which is used to take breath sample from the patient who has been delivered one or more than one drugs intravenously. The breathing circuit 102 may be directly connected to the patient's mouth or nose. In this configuration, it is called a mainstream breathing circuit. In a different configuration, the breathing circuit 102 may be connected to a separate tube, which is directly connected to the patient's mouth or nose. This configuration is called side stream configuration. One of the common intravenous drugs is propofol, which is a sticky molecule that tends to stick to the surface of the breathing circuit. To solve this problem, special material may be used to make the breathing circuit, for example, Teflon or special stainless steel. Heated breathing tube can also be used to reduce surface sticktion of propofol.

A sampling subsystem 104 is provided. The function of the sampling subsystem 104 is to sample the breath by pretreating the breath sample to improve measurement accuracy, and introduce the pretreated breath sample to sensors to measure gas composition and concentration of the sampled breath. The sampling subsystem 104 may have filters to remove or reduce unwanted substances in the breath, for example, water vapor in breath, interference compounds in breath, such as interference from different foods and odors in the stomach, mouth, esophagus and lungs. These interferences may lower the sensitivity and selectivity of the gas and vapor sensors used to detect target drug compounds. The sampling subsystem 104 may have pressure sensor to monitor the breathing pressure of the patient. The sampling subsystem may further comprise a pressure controller to provide the pressure level to system electronics to detect accurate breathing patterns of the patient. The calibration data is also generated and provided to the gas sensors and vapor sensors. The sampling subsystem 104 may have a temperature sensor to monitor the temperature of the breath from the patient. The subsystem 104 may further comprise temperature controller, or temperature feedback control loop. The temperature may be controlled at a required level and provided to the system electronics for data calibration or correction. The sampling subsystem 104 may have a flow sensor to detect the breathing flow rate of the patient. The signal can be used to detect the breathing pattern of the patient and for gas and vapor sensor calibration purpose. The sampling subsystem 104 may have a water trap to store water condensates from the breath sample.

Gas sensors 106 are provided to detect CO₂ and or O₂ concentration from breath. CO₂ concentration is an important parameter for breath measurement. The CO₂ concentration may be used to detect the end tidal of the breath. End tidal breath is considered the best part of breath for analysis. The end tidal breath is typically passed through the gas exchange process in lung. End tidal breath has the highest carbon dioxide concentration. In a normal human subject, this concentration is in the range from 4% to 5%. Early portions of the breath may contain gas in the dead volume of the air way, which does not participate in the gas exchange in lung. This portion of the breath is typically not used to measure the drug concentration. The CO₂ sensor may detect the end tidal breath. System electronics 108 may use this information to control sampling system to start sampling the end tidal breath. In an alternate embodiment, the drug sensor is constantly monitoring the drug concentration in breath. The system electronics 108 may extract the right concentration measurement at the same time when the CO₂ sensor detects the end tidal breath. Similarly, an O₂ sensor may be used for the same purpose. However, the CO₂ sensor is more commonly used. CO₂ sensor may also be used to provide real time monitoring of respiration condition of the patient undergoing anesthesia or other procedure. If abnormal CO₂ concentration is detected, an alarm may be generated to alert the doctor.

The intravenous drug sensor 110 may be a gas sensor or a vapor sensor depending on the drug being monitored. The sensor 110 measures the concentration of the target drug or drugs in the breath sample. For propofol, the typical concentration in the breath of patient undergoing intravenous anesthesia using propofol is from 0 ppb to 20 ppb. This requires the sensor 110 to be very sensitive and highly selective. The detection limit of sensor should be in the range of 0.1 ppb to 1 ppb, and the sensor 110 needs to only detect target drug without having response to all other potential gas compounds in the breath, for example, acetone, ethanol, isoprene, ammonia, methanol, pentane, ethane, etc.

The system electronics 108 may have interface circuit to different sensors and actuators, pumps to either receive sensor measurement data or submit signal for actuator, or pump operation. The system electronics 108 may have a power supply module to supply power to different parts of the whole system. The system electronics 108 may have a microprocessor or a microcontroller to receive, analyze, submit and store measurement and calibration data. The system electronics 108 may have memory device to store measurement data and calibration data. The system electronics 108 may have communication module to transmit and receive data with wired network or wireless network. A user interface 112 is used for user to input data for correlating or calculating the concentration of the anesthetic drugs in plasma using drug concentration in breath sample. The user may collect the output data from the user interface 112 by operating the system. A display 112 is used to display required information to the user. In some embodiments of the system, the user interface and the display device are operably liked to each other. In one embodiment, the user interface and the display device are present in one unit of subsystem (as 112). In another embodiment, the user interface and the display device are present in two separate subsystem.

The system monitors concentration of anesthetic drugs in the breath sample, which may be collected from an inhaled breath, an exhaled breath, or a combination of the two. The exhaled breath comprises various types of breath or gases depending on the sequence it comes out. At the beginning of exhalation, the breath coming out from the mouth and upper respiratory tracts (anatomically inactive part) of the respiratory system called “dead space”. This is followed by a plateau stage, wherein during an early part of the plateau stage, the breath comprises a mixture of dead space and metabolically active gases. The last portion of the exhaled breath comprises an end-tidal gas, which comes from the alveoli. In one example, the exhaled breath sample is collected at end-tidal breathing. Single or multiple samples may be collected for detecting anesthetic drugs. The breath sample may also comprise inspiratory gases. Inspiratory gases are the gases that patient inhaled during operation. The inspiratory gases may comprise synthesized air, or anesthesia gases. In some embodiments, the breath sample comprises end-tidal gas, gas from dead-space, inspiratory gas, or combinations thereof. In one embodiment, the breath sample comprises a mixed gas which may be a combination of end-tidal gas, gas from dead-space, and inspiratory gas.

When the drug is delivered at different dosage, the breath vapor concentration is correlated to the dosage, and the concentration may be back calculated to corresponding plasma concentration. The output plasma concentration may be used by the anesthesiologist to adjust the dosage to achieve the target plasma concentration more accurately than only relying on the pharmacokinetic model. The output plasma concentration may also help to prevent any operation error from the drug infusion system or human operation, increasing the safety of the intravenous anesthesia procedure. In some embodiments, the system further comprises a drug infusion device, wherein the plasma concentration of the drugs determined by the system is used to control the drug infusion device. In some examples, the measurement system also enables an automated close loop anesthetic drug delivering system by connecting the measurement system and the drug infusion system in a closed control loop. In some other examples, the measurement system also enables an automated open loop anesthetic drug delivering system. A reduced sensor offset determination may include measuring vapor concentration C1 before drug injection or infusion, and measuring vapor concentration Cv during operation. The breath vapor concentration is Cv-C1; C1 is the offset value from other interference gases or vapors from patient's breath or surrounding environment. The microcontroller may provide a breath by breath calculation of plasma concentration or an average plasma concentration over several breath.

The microprocessor measures the drug concentration in plasma using breath sample, wherein the measurement is based on the fact that the drug concentration in plasma may be correlated to the drug concentration in breath. In some embodiments, this correlation is represented by a transfer function. To monitor plasma concentration of intravenously delivered drugs, a transfer function is used to calculate the plasma concentration. The input of the transfer function includes at least measured drug concentration in exhaled breath of the patient. The output of the transfer function is the plasma concentration of the delivered drug. Other potential inputs to the transfer function may also be used to improve the accuracy of the calculation, for example, exhaled end tidal carbon dioxide concentration, exhaled pressure and flow rate, patient body temperature, patient body weight, age, gender, weight, height, BMI, or lung function parameters of a patient. In some embodiments, the format of the transfer function may be linear with only the first order terms. In some other embodiments, the format of the transfer function may be nonlinear with a second order or even higher order terms to achieve better calculation accuracy.

The concentration of the drug in the plasma is calculated and then compared with a target value. An alarm is triggered if the calculated concentration of the anesthetic drug in plasma is higher than the target value. If the value is within a target range, the procedure is repeated again starting from delivery of intravenous drug, as per the requirement of the procedure or user need. If a value of calculated drug concentration is out of the range of the target value, the procedure may be repeated starting, for example, from determination of the drug dosage.

Plasma drug concentration: Cₚ; Breath drug concentration: C_{b}, Exhaled end tidal CO₂ concentration: C_{co2}, Breathing flow rate: F_{b}, Patient body weight: W, Patient body temperature: T

### EXAMPLE 1

Cₚ=a·C_{b}+b eq (1)

In this example, the only input of the transfer function is C_{b} on the right side of the equation. The output of the transfer function is the plasma concentration of the drug Cₚ on the left side of the equation. “a” is a fitting parameter multiplied to C_{b}, and “b” is a fitting parameter to compensate for any offset between drug concentration in breath sample and drug concentration in plasma. The a and b are empirical numbers established from experiments, where the drug concentrations in breath sample are measured from patients. Linear regression fitting is used to extract the numerical value of fitting parameters a and b. Once a and b are established with enough statistical confidence, eq (1) may be used to predict plasma concentration of the target drug if the breath concentration of the drug is measured. Eq (1) is the simple transfer function with only first order terms. In real application, it provides the benefit of a simple numerical calculation, requiring less computing power and system memory to store fitting parameters.

### EXAMPLE 2

Cₚ=a·C_{b}+b·C_{b}²+c eq (2)

In this example, the input of the transfer is just the breath drug concentration C_{b} on the right side of the equation. The output of the transfer function is the plasma concentration of the drug Cₚ on the left side of the equation. a is a fitting parameter multiplied to C_{b}, b is the second order fitting parameter multiplied to the square of the breath drug concentration, and c is a fitting parameter to compensate for offset. The fitting parameters are established empirically. One difference between eq (2) and eq (1) is the addition of a second order term, which provides better prediction accuracy but typically requires more computing power and data storage space.

### EXAMPLE 3

Cₚ=[(a·C_{b})/C_{co2}]+b. eq (3)

In this example, the inputs of the transfer function are the breath drug concentration C_{b} and the exhaled end tidal carbon dioxide concentration C_{CO2} on the right side of the equation. The output of the transfer function is the plasma concentration of the drug Cₚ on the left side of the equation. a is a fitting parameter multiplied to the division product of the breath drug concentration to the end tidal carbon dioxide concentration. b is a fitting parameter to compensate for offset. Both a and b are empirical fitting parameters extracted from measured plasma drug concentration, breath drug concentration and end tidal carbon dioxide concentration. Once fitting parameters a and b are established with enough statistical confidence, eq (3) may be used to predict plasma drug concentration with the input of measured breath drug concentration and end tidal carbon dioxide concentration. In this transfer function, end tidal carbon dioxide concentration is used to normalize measured breath drug concentration. Normalization reduces the prediction error between different patients from their different respiration condition. Patients with higher end tidal carbon dioxide concentration may have better gas exchange efficiency and therefore higher exhaled drug concentration with the same delivered dosage with a patient with lower exhaled carbon dioxide concentration. Another benefit of using carbon dioxide concentration is that, if there is any dilution effect from the sampling or measurement process, the same dilution effect may occur with carbon dioxide concentration as well. Therefore, using carbon dioxide concentration to normalize the drug concentration reduces the measurement variation due to these effects.

For example, propofol with same dosage is intravenously delivered to two patients having identical weight. One patient has a higher end tidal exhaled carbon dioxide concentration around 5%. The other patient has a low end tidal carbon dioxide concentration around 4.5%. This means the first patient has better gas exchange efficiency in his lung than the second patient. Although their plasma drug concentrations are the same, their exhaled drug concentration may be different due to their lung gas exchanging difference. With the same plasma concentration, the first patient may have a 10% higher breath drug concentration than the second patient. Therefore, by using eq (1) to predict plasma concentration, there is a 10% difference between the two patients. This shows that eq (1) does not give accurate plasma concentration values if there is variation in patient's lung gas exchange rate. However, using exhaled carbon dioxide concentration to normalize the breath drug concentration to predict plasma concentration using eq (3), the error can be eliminated.

### EXAMPLE 4

Cₚ=[(a·C_{b})/(b·C_{co2}+c·F_{b})+d. eq (4)

In this example, patient breathing flow rate is also used as an input to the transfer function. Sensing technologies that are used to measure gas concentration are typically flow rate dependent. Adding flow rate as an input to the transfer function may reduce measurement variation introduced from breathing flow rate variations. Eq (4) is just one example showing how flow rate may be incorporated in the transfer function. Flow rate may also be incorporated in other ways.

### EXAMPLE 5

Cₚ=a·C_{b}/W+b eq (5)

In this example, patient body weight is used as an input to the transfer function. Body weight is used in pharmacokinetic models to calculate the right drug dosage in many intravenous drug delivery practices. For example, recommended dosage for propofol is: for initial Bolus: 0.8-1.2 mg/kg; for infusion: start at 140-200 μg/kg/min, at 10 min: 100-140 μg/kg/min, after 2 hours: 80-120 μg/kg/min. Body weight is proportional to the blood volume of a patient. Therefore, it is also often an important parameter for drug concentration in blood or plasma and the drug concentration in breath sample. Using patient body weight as an input parameter may potentially normalize prediction error from body weight variation of different patients.

### EXAMPLE 6

Cₚ=a·C_{b}·e^{(T/T0)β}+b eq (6)

In this example, patient body temperature is used as an input to the transfer function. The volatility of a drug compound is dependent on the body temperature. The higher the body temperature, the higher is the breath drug concentration. By incorporating body temperature into the transfer function, eq (6) may reduce temperature variation that causes prediction error of plasma drug concentration.

The given examples are non-limiting examples of potential transfer functions that may be used to calculate drug concentration in plasma based on measured values of drug concentration in breath, end tidal carbon dioxide concentration, breathing flow rate, body weight, or body temperature. Other transfer functions may be formed by using given transfer function examples to incorporate all or a sub set of these inputs. Additional inputs may be included. These inputs may be the physiological conditions of the patient, environmental parameters or measurement system and components related parameters, among others.

One or more other examples may be used to obtain accurate end-tidal propofol values. By adding a CO₂ sensor to the mixing chamber in which the mixed propofol concentration is measured, the end-tidal concentration of propofol may easily and accurately be solved. In the following, Cx is the mixed expired concentration measured in the mixing chamber, cx(t) is the expired concentration as a function of time, and c^{et}x is the end-tidal concentration of either x=propofol or x=CO₂. V_{mixed} is the volume of the mixing chamber and f(t) is the expired flow as a function of time. Sampling for the mixing chamber can be done either from the D-lite (on common sampling point) or from the expiratory limb of the breathing circuit (two sampling points; one for the gas module and another for the mixing chamber). In both of these examples,

∫ₑₓₚf(t)c_{CO2}(t)dt=a′·V_{mixed}C_{CO2} eq (7)

∫ₑₓₚf(t)c_{PRO}(t)dt=a′·V_{mixed}C_{PRO} eq (8)

where a′ is a constant that depends on the sampling flow. The exhaled CO₂ and propofol curves are assumed to have the same shapes so that they differ only by a constant factor k. This is a feasible assumption if there is no propofol in the inhaled gas. This is typical at least in the intensive care unit (ICU) respirators with an open circuit; perhaps also in the anesthesia machines, where propofol gets absorbed. In this case:

c_{PRO}(t)=k·c_{CO2}(t) eq (9)

and therefore also for the end-tidals

c_{PRO}^{et}=k·c_{CO2}^{et} eq (10)

From eqns. (7)-(9) for the mixed concentrations:

C_{PRO}=k·C_{CO2} eq (11)

From eqns (10) and (11), a simple equation for the end-tidal propofol concentration is derived as:

$${c}_{PRO}^{et} = \frac{{C}_{PRO} {c}_{CO 2}^{et}}{{C}_{CO 2}} \qquad eq ( 12 )$$

The measurement of the concentrations of propofol and CO₂ in the mixing chamber, and the end-tidal CO₂ is significant, however in some cases accurate measurement of the flow may not require dependence on the user's need. The need to synchronize and integrate flow with the CO₂ concentration is avoided, a step that is prone to introduce errors.

The basic assumption for eqn. (9) is not valid, for example, if one of the two gases is more strongly absorbed in the airways or tubings, then it is not possible to correct for the deadspace. Therefore, the end-tidal portion of the expired propofol utilizing a valve is required to be processed for further detection. Controlling the valve for accurate measurement is desirable. The pressure and flow signals are not in synchrony with the gases; the measured CO₂ curve of the gas module is not in synchrony either. The time delays are not constants but rather depend on the dynamic pressure variations so synchronization may be somewhat cumbersome but not impossible.

The easiest solution might again be to add a second CO₂ sensor close to the opening valve of the mixing chamber and use this CO₂ signal to open and close the valve that lets in the end-tidal portion of the expired gas. This requires of course that this signal may be obtained and processed fast enough. Again, sampling may be done either from the D-lite or from the expiratory limb. One sampling point may be preferred with one gas module that handles all measurements.

In one or more examples, the method provides a safety alarm if the concentration of anesthetic drug is higher than a safety threshold value preset by the anesthesiologist. The “safety threshold value” means a threshold value of the anesthetic drug concentration which is safe for the patient undergoing anesthesia procedure. In some examples of the method, the monitoring of anesthetic drug concentration in plasma is a continuous real time process. In this example, the real time anesthetic drug concentration in plasma helps the anesthesiologist to adjust the drug dosage.

To determine a dosage regimen for an anesthetic drug delivered to a patient is significant for delivery rate of the drug to achieve a desired pharmacologic effect for the patient while any associated side effects are minimized. Some of the anesthetic drugs have a close relationship between their dosage regimen, for example propofol, remifentanil, and afentanil. The administration of the drug based on the dosage regimen on the pharmacokinetic model may be improved. In another example, the concentration of drug in plasma may be used in conjunction with a pharmacokinetic model to provide correction to the pharmacokinetic predication of anesthetic drug concentration in plasma. Using a computer with a pharmacokinetic program permits control of a desired plasma concentration of an agent, such as propofol. Target controlled infusion is one of the methods for administering an intravenous anesthesia agent using a computer to control the infusion pump.

In accordance with one or more embodiments of the system, the anesthetic drug concentration is determined after direct administration of the drug into a patient's blood stream, rather than administering through a breathing circuit. In some examples, the administered anesthetic drug is bound to proteins or absorbed into fat, and the bound or absorbed drug does not produce a pharmacological effect. In one or more examples, a portion of the bound drug may exist in equilibrium with an unbound drug. In some examples, the drug may exist in a free form. Drug metabolism typically precedes clearance of the drug from the bloodstream and termination of its effect. The effect of the drug may also be terminated by the excretion of the free drug in the urine, digestive tract or in exhaled breath. The concentration of an anesthetic agent in the body depends on the amount of anesthetic agent administered and the amount of the agent eliminated from the body over a given period of time. The concentration indicates a characteristic of metabolism of the agent in the patient's body.

The intravenously delivered drug may be selected from, but is not limited to, an analgesic drug, an amnesia drug, a muscle relaxation drug or a chemotherapeutic drug. An example of an anesthetic drug is propofol, which is widely used as a short acting intravenous anesthetic agent, hydrophobic and volatile in nature. The propofol is administered as a constant intravenous infusion to deliver and maintain a specific plasma concentration. The clearance of propofol from the body is controlled by metabolic processes, primarily through the liver.

In one or more embodiments of the systems, the system is specifically used for monitoring a propofol using a patient's breath. In some embodiments, the system provides a more accurate measurement of anesthetic drug concentration in plasma than pharmacokinetic models. Use of a multi-parameter transfer function is more accurate and robust method than other breath based measurement. The system only uses the concentration of components or drugs in breath sample as input parameter to calculate a concentration of drug in plasma.

In some embodiments, the system employed breath sample that comprises end-tidal gas, gas from dead-space, inspiratory gas, or combinations thereof. The propofol concentration in the breath sample comprises mixed gases, such as combination of end-tidal gas, gas from dead-space, and inspiratory gas, is easier using available sensors. The propofol concentration in the end tidal gas is determined suing the system by determining the concentration of another gas in the end tidal gas, and also by assuming a ratio of the concentration of propofol and another gas in the end-tidal gas and the ratio of the concentration of propofol and another gas in the breath sample comprises mixed gases are same. For example, the end-tidal concentration of propofol measurement may be difficult because of unavailability of a fast sensor that may measure the very low concentration of propofol in end tidal gas. Instead, the concentrations of propofol and another gas in the mixed gas sample is easily measurable. The measurement of the end-tidal concentration of another gas, such as CO₂ may be easier as fast 10ms sensors are available. The end tidal concentration of propofol may be determined by making an assumption of equal ratios of propofol and CO₂ in mixed gases and in the end tidal gas as described above. Therefore, the plasma concentration of propofol is determined using the propofol concentration in the end-tidal gas and using the above assumption.

The scope of the invention is defined by the claims, and may comprise other examples not specifically described that would occur to those skilled in the art. Such other examples are intended to be within the scope of the claims.

1. A system for monitoring a concentration of an anesthetic drugs using a patient's breath, comprising:
a sampling subsystem for processing the patient's breath to form a breath sample;
one or more sensors to measure drug concentration in the breath sample;
one or more sensors to measure a concentration of gases in the breath sample; and
one or more microprocessors for determining a concentration of the drugs in a plasma of the patient using a transfer function and the concentration of the drug in the breath sample.

2. The system of claim 1, further comprises a user interface and a display device operatively coupled to the microprocessors.

3. The system of claim 1, wherein the sampling subsystem comprises a breath sample conduit and a heating element that heats the conduit.

4. The system of claim 1, wherein the sampling subsystem comprises two or more devices for filtration, breath pressure control, breath flow rate control, breath temperature control, or normalizing vapor density.

5. The system of claim 1, wherein the sampling subsystem is operated periodically or continuously.

6. The system of claim 1, wherein the sensor is selected from two or more of the pressure sensors, flow rate sensors, humidity sensors, temperature sensors, gas sensors or drug vapor sensors.

7. The system of claim 1, wherein the drug vapor sensor detects propofol in the patient's breath sample.

8. The system of claim 1, wherein the gases comprise oxygen, carbon dioxide, or both.

9. The system of claim 1, wherein the gases comprise one or more metabolites of delivered drug in the breath sample.

10. The system of claim 1, wherein the anesthetic drug comprises propofol.

11. The system of claim 1, wherein the transfer function has an input and an output value.

12. The system of claim 11, wherein the input value of the transfer function depends on the anesthetic drug concentration in an exhaled end tidal breath, carbon dioxide concentration in the exhaled end tidal breath, pressure of the exhaled breath, flow rate of the exhaled breath, the patient's body temperature, the patient's body weight, the patient's gender, age of the patient, body mass index (BMI) of the patient, lung function of the patient, or combinations thereof.

13. The system of claim 11, wherein the input value of the transfer function depends on at least the measured anesthetic drug concentration in the exhaled end tidal breath of the patient.

14. The system of claim 11, wherein the output value of the transfer function is the plasma concentration of the delivered drug.

15. The system of claim 1, wherein the plasma concentration of the drugs triggers an alarm.

16. The system of claim 1, wherein the plasma concentration of the drugs is used to control a drug infusion device.

17. The system of claim 1, wherein the transfer function comprises a linear equation or a non-linear equation.

18. The system of claim 17, wherein the transfer function comprises a non linear equation that uses a second order or higher order.

19. The system of claim 1 is a continuous real time process.

20. A system for monitoring a concentration of propofol using a patient's breath, comprising:
a sampling subsystem for processing the patient's breath to form a breath sample;
one or more sensors to measure propofol concentration in the breath sample;
one or more sensors to measure a concentration of gases in the breath sample; and
one or more microprocessors for determining a concentration of the propofol in a plasma of the patient using a transfer function and the concentration of the propofol in the breath sample.

21. The system of claim 20, wherein the transfer function depends on propofol concentration in exhaled end tidal breath, carbon dioxide concentration in exhaled end tidal breath, pressure of exhaled breath, flow rate of exhaled breath, patient's body temperature, patient's body weight, patient's gender, age of a patient, body mass index (BMI) of a patient, lung function of a patient, and combinations thereof.

22. The system of claim 20, wherein the sensors comprise at least one sensor for measuring propofol concentration and at least one sensor for measuring other gases.

23. The system of claim 20, wherein the breath sample comprises end-tidal gas, gas from dead-space, inspiratory gas, or combinations thereof.

24. The system of claim 23, wherein the sensors measure the concentration of propofol and the concentration of at least another gas in the breath sample.

25. The system of claim 24, wherein the propofol concentration in the end tidal gas is determined by determining the concentration of the another gas in the end tidal gas and assuming a ratio of the concentration of propofol and another gas in the end-tidal gas and the ratio of the concentration of propofol and another gas in the breath sample are same.

26. The system of claim 25, wherein the plasma concentration of propofol is determined using the propofol concentration in the end-tidal gas.
